(19) United States
(12) Patent Application Publication
(10) Pub. No.: US 2024/0418657 A1
(43) Pub. Date: Dec. 19, 2024

(54) PROCESS AND APPARATUS FOR CHECKING A GAS MEASURING DEVICE

(71) Applicant: Dräger Safety AG & Co. KGaA, Lübeck (DE)

(72) Inventor: Sebastian DRAACK, Lübeck (DE)

(21) Appl. No.: 18/738,151

(22) Filed: Jun. 10, 2024

(30) Foreign Application Priority Data
Jun. 14, 2023 (DE) 10 2023 115 474.5

(51) Int. Cl. G01N 23/06

(52) U.S. Cl. CPC G01N 23/06

(57) ABSTRACT

A verification process and a verification device verify a gas measuring device (100). A radiation source (1) emits radiation into a measurement chamber (2), the intensity of the emitted radiation being described by an input signal [x_{Ref}(t), x_{Üb}(t)]. A detector (4) generates an output signal [y_{Ref}(t), y_{Üb}(t)] depending on the intensity of radiation in the measurement chamber. The gas measuring device is assumed to be intact in a reference period (Ref_ZR). The objective is to check whether it is also intact in a verification period (Üb_ZR). In both periods, an indicator of system behavior of a system model is calculated in each case. This system model is excited with the input signal and provides the measured output signal in response to the excitation. The two system behavior indicators are compared with each other. Depending on this comparison, measuring device status information in the verification period is derived.

### CROSS REFERENCE TO RELATED APPLICATIONS

This application claims the benefit of priority under 35 U.S.C. § 119 of German Application 10 2023 115 474.5, filed Jun. 14, 2023, the entire contents of which are incorporated herein by reference.

### TECHNICAL FIELD

The invention relates to a process and a device for automatically checking a gas measuring device (gas detection device/gas detector). Such a gas measuring device is able to check a spatial region (special area/space) for the presence of at least one target gas to be detected and/or to determine the concentration of such a target gas.

### BACKGROUND

Gas measuring devices comprising a radiation source and a measurement chamber have become well known. A gas sample to be analyzed enters the measurement chamber. Radiation emitted by the radiation source penetrates the measurement chamber. A target gas to be detected absorbs part of the radiation that passes through the measurement chamber. A detector in or on the measurement chamber measures the intensity of the incident radiation. This intensity is an indicator (a measure) of the target gas concentration. In one embodiment of the invention, a gas measuring device, which can be tested according to the invention, also uses this principle.

Such a target gas can pose a health risk to humans. It is therefore imperative that the gas measuring device reliably detects a harmful target gas. On the other hand, it is desirable that the gas measuring device generates relatively few false alarms. For both reasons, it is necessary to check the gas measuring device from time to time.

### SUMMARY

It is an object of the invention to provide a verifying process and a verifying device which make it possible to test a gas measuring device with a radiation source, a measurement chamber, and a detector with less effort than known processes and devices.

The object is attained by a verification process with features disclosed herein and by a verification device with features disclosed herein. Advantageous embodiments are disclosed herein. Advantageous embodiments of the verification process according to the invention are, where appropriate, also advantageous embodiments of the verification device according to the invention and vice versa.

The gas measuring device to be tested comprises a measurement chamber. This measurement chamber can hold a gas sample to be tested. During use, the gas sample originates from a spatial region (area) to be monitored. Furthermore, the gas measuring device to be checked comprises a radiation source and a detector. The radiation source is capable of emitting radiation into the measurement chamber. This radiation is in particular electromagnetic radiation, e.g. infrared radiation, or sound (acoustic radiation, in particular ultrasound). The radiation penetrates the measurement chamber at least once, optionally several times.

The detector is able to generate an output signal depending on the intensity of the impinging radiation. The output signal refers to the intensity of the radiation after the radiation has penetrated (passed through) at least once through at least part of a gas sample in the measurement chamber. The radiation intensity is therefore changed by a target gas to be detected. The output signal generated by the detector therefore correlates with the concentration of a target gas in a gas sample, whereby the gas sample is taken up by the measurement chamber.

The verification process according to the invention automatically verifies such a gas measuring device. The verification device according to the invention is able to verify such a gas measuring device.

It is assumed that the gas measuring device is intact (faultless/in good order) during a reference period. At least the radiation source, the measurement chamber and the detector are intact, preferably also an optional evaluation unit and an optional own power supply unit. It is checked whether the gas measuring device is also intact in a verification (test) period. As a rule, the verification period is after the reference period. However, it is also possible that the reference period is after the verification period and the gas measuring device is to be checked afterwards.

The following steps are carried out in both the reference period and the verification period:
There is a gas sample in the measurement chamber.
The radiation source emits radiation into the measurement chamber.
An indicator of the intensity of the emitted radiation is specified or measured. This indicator refers to the intensity before the radiation enters the measurement chamber and is used as an input signal. The input signal can be constant over time or vary over time.
The emitted radiation penetrates at least once the measurement chamber at least part of a gas sample, optionally several times to extend the optical path. The intensity of the radiation after penetrating the gas sample depends on the input signal and the gas sample in the measurement chamber.
The detector generates an output signal. This output signal depends on the intensity of the radiation and therefore on the input signal. The output signal refers to the intensity of the radiation after the radiation has passed at least once through at least part of the gas sample in the measurement chamber.
An indicator of the system behavior of a system model (emulator system/theoretical system) is calculated, either in the time domain or in the frequency domain. This system model is excited with the input signal and generates the output signal in response to this excitation.

An indicator of a reference system behavior is calculated as the indicator of the system behavior of the system model that is excited with the specified or measured input signal in the reference period and generates the measured output signal in response to this excitation. The reference system behavior therefore refers to an intact gas measuring device. Accordingly, an indicator of a system behavior of the system model that is excited with the specified or measured input signal during the verification period and generates the measured output signal in response to this excitation is calculated as the indicator of the verification system behavior. As mentioned above, the input signal describes the intensity of the emitted radiation before this radiation reaches the measurement chamber, and the output signal describes the intensity of the radiation after the radiation has penetrated the gas sample. Preferably, the output signal refers to the intensity of the radiation after this radiation has completely penetrated the measurement chamber at least once.

According to the invention, the two behavior indicators are compared with each other, i.e. the verification system behavior indicator is compared with the reference system behavior indicator. Depending on the result of the comparison, status information about the status of the gas measuring device in the verification period is derived.

The invention can be used each time the gas measuring device needs to be checked. In many cases, the invention eliminates the need to take the gas measuring device out of service after a predetermined period of time, regardless of its condition.

In many cases, the invention makes it possible to detect both an error of the radiation source and an error of the detector. More specifically, the event can be detected that the radiation source and/or the detector acts different in a relevant way in the verification period than in the reference period. In particular, a fault in the radiation source can be detected if a predefined input signal is used and the radiation source should follow this input signal. If the radiation source is faulty, the actual intensity of the radiation usually deviates considerably from the input signal. A fault in the measurement chamber can also be detected, for example condensation of liquid on a wall of the measurement chamber. Preferably, a fault of an optional evaluation unit and a fault of an optional separate power supply unit of the gas measuring device can also be detected. Both a fault that occurs suddenly, for example due to an external influence, and a fault that occurs gradually due to ageing or parameter drift can be detected thanks to the invention. This advantage is achieved because in many cases both a faulty radiation source and a faulty detector as well as a faulty measurement chamber lead to a significantly changed system behavior and thus also to a significantly changed indicator of the system behavior. The comparison therefore leads to a deviation between the two system behavior indicators, whereby the deviation is greater than a specified tolerance.

According to the invention, the verification system behavior is compared with the reference system behavior. The verification system behavior describes the reaction of the system model in the verification period to an excitation by the radiation source, the reference system behavior describes the reaction of the same system model in the reference period to the same excitation. The same device is examined in both of these processes. Of course, the terms “same excitation” and “same system model” only apply if the gas measuring device is also intact during the verification period. In other words, the verification system behavior describes the state of the gas measuring device in the verification period, while the reference system behavior describes the state of the gas measuring device in the reference period. In particular, the same device and therefore the same radiation source, the same measurement chamber and the same detector are used in both periods. If the radiation source, the measurement chamber, and the detector are also intact in the verification period, the verification system behavior ideally matches the reference system behavior. Preferably, a tolerance is specified and the radiation source, the measurement chamber and the detector are considered as intact if the two system behavior indicators do not deviate from each other by more than the tolerance. This tolerance takes into account an unavoidable measurement error as well as possible measurement noise and process noise.

The reference system behavior is recorded in a state in which the gas measuring device—or at least the radiation source, the measurement chamber, and the detector—is intact. In many cases, it is sufficient for the following two conditions to be met:
In the reference period and in the verification period, two gas samples are used which absorb the penetrating radiation with sufficient coincidence. This is usually the case, in particular, if the two gas samples match chemically with sufficient accuracy.
The input signal, i.e. the emitted radiation, is sufficiently broadband so that an error of the device can also be detected which error only occurs in a sub-range (portion) of the frequency band.

Thanks to the invention, it is often not necessary to ensure that other requirements are met, in particular that the gas sample used in the reference period has a specific chemical composition. Furthermore, it is often not necessary to control the radiation source in the verification period and in the reference period with the same time characteristic. In particular, it is not necessary for the respective electrical voltage applied to the voltage source to match in both time periods. This advantage is achieved because, according to the invention, two system behaviors of two system models are calculated and compared with each other and these two system behaviors match sufficiently accurately with intact components of the gas measuring device even if different controls and therefore different input signals are used in the two time periods.

The verification system behavior matches the reference system behavior at least to an unavoidable tolerance if the following conditions are met with sufficient accuracy:
The gas measuring device is also intact during the verification period.
The gas sample that is in the measurement chamber during the verification period corresponds sufficiently accurately to the gas sample that is in the measurement chamber during the reference period.
In both periods, approximately the same ambient conditions act on the measurement chamber.

Preferably, an alarm is generated if the two system behavior indicators deviate from each other by more than a specified tolerance. One reason of a deviation is a defect in a component of the gas measuring device. This defect may have occurred suddenly, for example due to a failure, or gradually, for example due to the drift of a parameter, in particular due to ageing. Preferably, an alarm can be used as a reason to examine the gas measuring device more closely.

The invention does not require that the same gas sample is used in both periods and that the same ambient conditions act on the measurement chamber. Even if the gas samples or the ambient conditions differ from each other, the gas measuring device may be wrongly classified as defective. Even in this case, however, an actually defective gas measuring device is usually recognized as defective.

The tolerance is an adjustable parameter. If the two system behavior indicators deviate from each other by more than this tolerance, the gas measuring device is classified as faulty. It is possible that this tolerance is specified depending on possible changes in the ambient conditions. This configuration reduces the risk that a significant change in ambient conditions could lead to the gas measuring device being wrongly classified as faulty.

In contrast to many known processes of verification, the invention does not require that a gas sample used in the reference period has a composition known in advance.

The invention does not require that the input signal in the verification period corresponds exactly to the input signal in the reference period. The invention also does not essentially require that it is determined in advance how a possible interference signal affects a signal of the detector and/or is contained in a signal of the detector. These effects are achieved because two indicators of system behavior are compared with each other.

According to the invention, the detector generates an output signal in each of the two time periods. This output signal is an indicator of the intensity of the radiation after the radiation has penetrated a gas sample in the measurement chamber. Various configurations are possible as to which indicator the detector measures in each of the two time periods.

In one embodiment, the emitted radiation strikes (impinges on) the detector after the radiation has completely penetrated the measurement chamber at least once. The detector is configured as a photodetector and generates the output signal depending on the intensity with which the radiation strikes the photodetector. In this configuration, the output signal is relatively insensitive to ambient conditions such as temperature, pressure, humidity, or vibrations. In another embodiment, the emitted radiation triggers a physical effect in the measurement chamber. For example, the radiation changes the temperature and/or the pressure in the measurement chamber or generates an acoustic effect. The detector measures an indicator of the physical effect triggered by the radiation. In many cases, this configuration is relatively insensitive to the light intensity in the surroundings.

According to the invention, in each of the two time periods a respective indicator of the system behavior of the system model is calculated. In a preferred embodiment, the step of calculating the indicator for the system behavior comprises in each of the two time periods the following step: an indicator for the cross-correlation between the input signal and the output signal is calculated.

The input signal and the output signal are time-resolved signals, i.e. signals in the time domain. As a rule, the cross-correlation is therefore also calculated in the time domain. In a further development of this embodiment, the respective cross-correlation is transformed from the time domain into the frequency domain for both time periods, in particular by a Fourier transformation or another Laplace transformation. As a result of the transformation, preferably the respective transfer function of the system model in the complex (complex domain) is calculated. A reference transfer function describes the system behavior in the reference time period, a verification transfer function describes the system behavior in the verification time period. The transfer function in the complex can sometimes also be calculated without using a cross-correlation.

In one embodiment, the verification process according to the invention is carried out using a signal processing unit. This signal processing unit calculates the respective indicator for the system behavior both in the reference time period and in the verification time period, compares them with each other and supplies the status information about the gas measuring device via the gas measuring device.

In one implementation, this signal processing unit is a component of the gas measuring device to be checked. The gas measuring device is therefore able to check itself. In another embodiment, the signal processing unit is a component of a computer unit. This computer unit is spatially remote from the gas measuring device. A data connection is established at least temporarily between the gas measuring device and the computer unit. This configuration makes it easier to provide a powerful signal processing unit. This is particularly advantageous if the gas measuring device is a portable device, especially a portable device that a user carries with him/her. The computer unit can be configured as a stationary device.

According to the invention, the gas measuring device comprises a measurement chamber and a detector. In one embodiment, the gas measuring device additionally comprises a reference chamber and a reference detector. While the measurement chamber is filled with a gas sample, at least during the verification period, which may contain the or at least one target gas to be detected, the reference chamber is filled with a reference gas which is ideally free of the or each target gas to be detected. Preferably, the reference chamber is fluid-tightly sealed against the measurement chamber. The two chambers are arranged in parallel with respect to the emitted radiation. Therefore, the radiation emitted by the radiation source penetrates at least once each both the measurement chamber and the reference chamber. The radiation therefore penetrates the gas sample in the measurement chamber and the reference gas in the reference chamber. This applies to both the reference period and the verification period.

According to the invention, the detector generates a time-resolved signal, namely the output signal, depending on the intensity of the emitted radiation in the measurement chamber. This radiation has preferably completely penetrated the measurement chamber at least once. According to the embodiment just described, the reference detector generates a time-resolved reference signal depending on the radiation emitted in the reference chamber. This radiation has preferably completely penetrated the reference chamber at least once.

The configuration with the output signal from the detector and the reference signal from the reference detector makes it possible to compare these two signals with each other and thereby detect a target gas or rule out the presence of a target gas. The target gas or each target gas only affects the measurement chamber, but ideally not the reference chamber. Ambient conditions, in particular the ambient temperature and humidity, as well as vibrations and other external influences, however, affect both chambers.

According to the invention, an indicator of the system behavior of a system model is calculated both in the reference period and in the verification period. This system model is excited with the respective input signal and provides the output signal as a reaction to the excitation. According to the embodiment just described, the signal generated by the reference detector after radiation has passed through the reference chamber at least once is used as the input signal. This applies both to the step of calculating the reference system behavior indicator and to the step of calculating the verification system behavior indicator.

In many cases, this configuration compensates to a certain extent for the influence of ambient conditions on the respective system behavior. It is often not necessary to create exactly the same environmental conditions in the reference period and the verification period.

The gas measuring device is checked by the verification (checking) process according to the invention and the verification device according to the invention. In one embodiment, the invention is used to adjust and/or calibrate the gas measuring device. According to this embodiment, the output signal depends on the intensity of the radiation in the measurement chamber, preferably on the intensity after this radiation has penetrated the measurement chamber, and additionally on the applied value of a parameter. The system model, which is excited by the input signal and provides the output signal in response to the excitation, also depends on the applied parameter value. For example, the output signal comprises an indicator of the concentration of a gas to be detected, whereby this concentration can vary over time. The parameter describes a functional relationship between an output signal of the detector and the target gas concentration and is, for example, a proportionality factor.

According to the invention, the reference system behavior is calculated in the reference period. According to the embodiment with the parameter, several possible values are specified for the parameters. Each possible parameter value leads to a system model. For each possible parameter value, the respective verification system behavior of this system model is calculated in the verification period and compared with the previously calculated reference system behavior. A comparison result is then calculated for each possible parameter value. Which value for the parameter is actually applied is determined depending on this comparison result. For example, the value that leads to the smallest deviation between the reference system behavior and the verification system behavior is applied for the parameter.

In many cases, this embodiment makes it easier to adjust the gas measuring device. It is often not necessary to use a calibration gas sample with a specific chemical composition that is known with sufficient precision, whereby the gas measuring device to be calibrated analyzes this calibration gas sample. However, this embodiment can also be used in conjunction with a calibration gas sample.

The invention further relates to a measuring process and a measuring arrangement for measuring the concentration of at least one target gas. The process is carried out using a gas measuring device, and the arrangement comprises a gas measuring device which is constructed as just described, i.e. comprises a measurement chamber, a detector, and a radiation source. According to one embodiment, a verification procedure according to the invention is carried out at least once for this gas measuring arrangement. The measuring arrangement comprises a verification device according to the invention, which is configured to verify the gas measuring device.

The invention is described below by means of an embodiment examples. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a graph showing the transmittance of methane as a function of the wavelength;

FIG. 2 is a graph showing the transmittance of propane as a function of the wavelength;

FIG. 3 is a schematic view of the gas measuring device in the reference period;

FIG. 4 is a schematic view of the signal processing unit of FIG. 3 in detail;

FIG. 5 is a schematic view of the gas measuring device during the verification period;

FIG. 6 is a schematic view of the signal processing unit in FIG. 5 in detail;

FIG. 7 is a schematic view of a different configuration of the gas measuring device with a measurement chamber and additionally a reference chamber in the reference period;

FIG. 8 is a schematic view of the configuration of FIG. 7 during the verification period; and

FIG. 9 is a schematic view of a different embodiment in which the signal processing unit is a component of a spatially remote computer unit.

### DESCRIPTION OF PREFERRED EMBODIMENTS

Referring to the drawings, in the example embodiments, the invention is used to check a gas measuring device. This gas measuring device is used or can be used to check a spatial region for several different combustible target gases. In one embodiment, the respective concentration of each target gas is determined, and preferably information about at least one determined target gas concentration is output in a form perceptible by a human. In another embodiment, an alarm is output if the measured concentration of at least one target gas is above a predetermined threshold.

The gas measuring device to be tested utilizes a principle known from the prior art, namely the following: Various target gases attenuate electromagnetic and/or acoustic radiation. One indicator of the attenuation is the spectral response, expressed as the transmittance Tr in %, as a function of the wavelength λ of radiation. The lower the transmittance Tr[λ], the more the target gas attenuates the radiation.

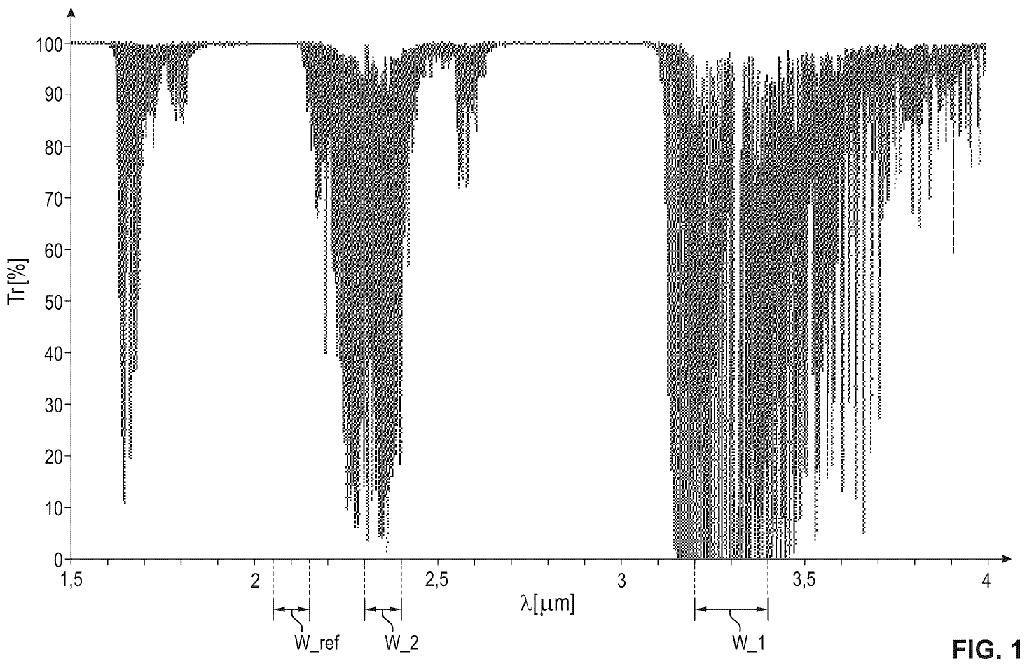

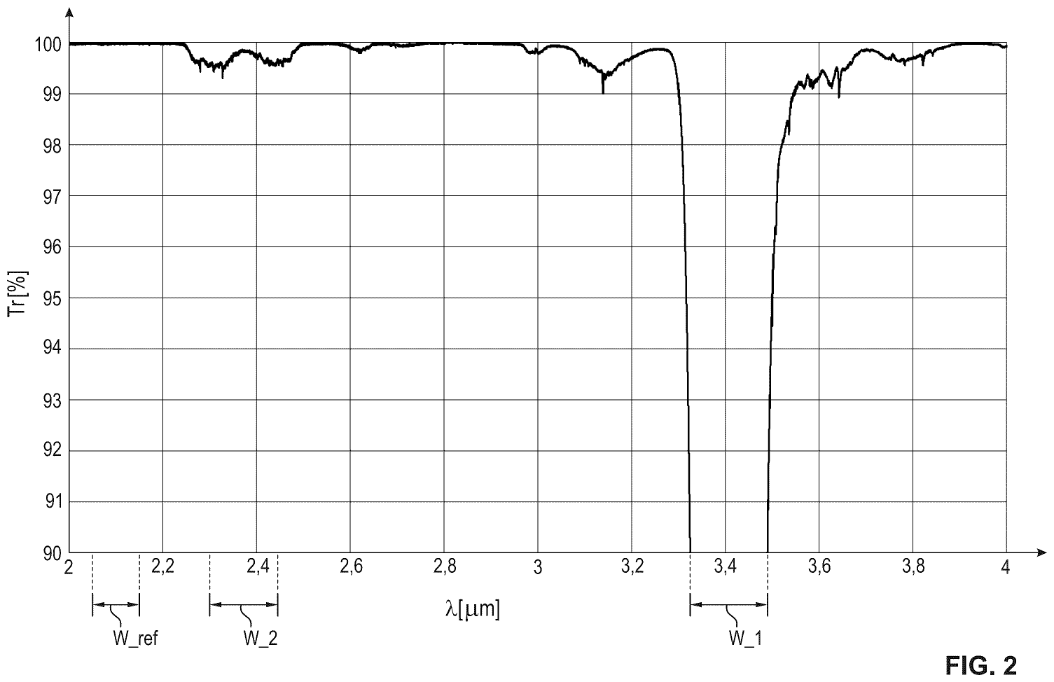

FIG. 1 shows an example of the spectral curve for the hydrocarbon methane and FIG. 2 for propane, in each case at an exemplary concentration of methane or propane. In this example, electromagnetic radiation penetrates a gas sample in a measurement chamber. The wavelength λ in micrometers [μm] is plotted on the x-axis and the transmittance Tr in percent [%] on the y-axis.

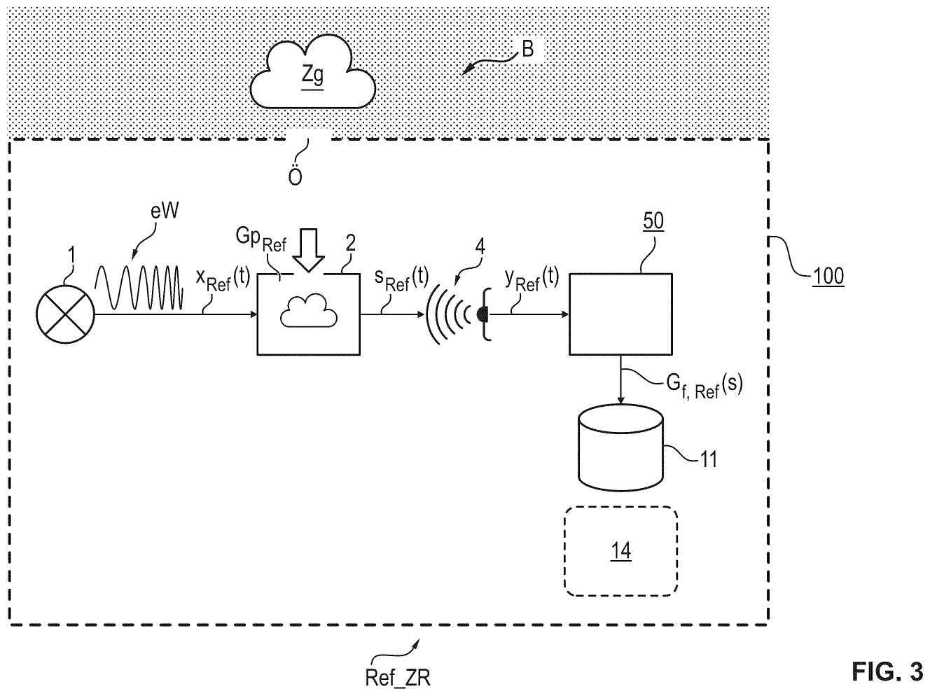

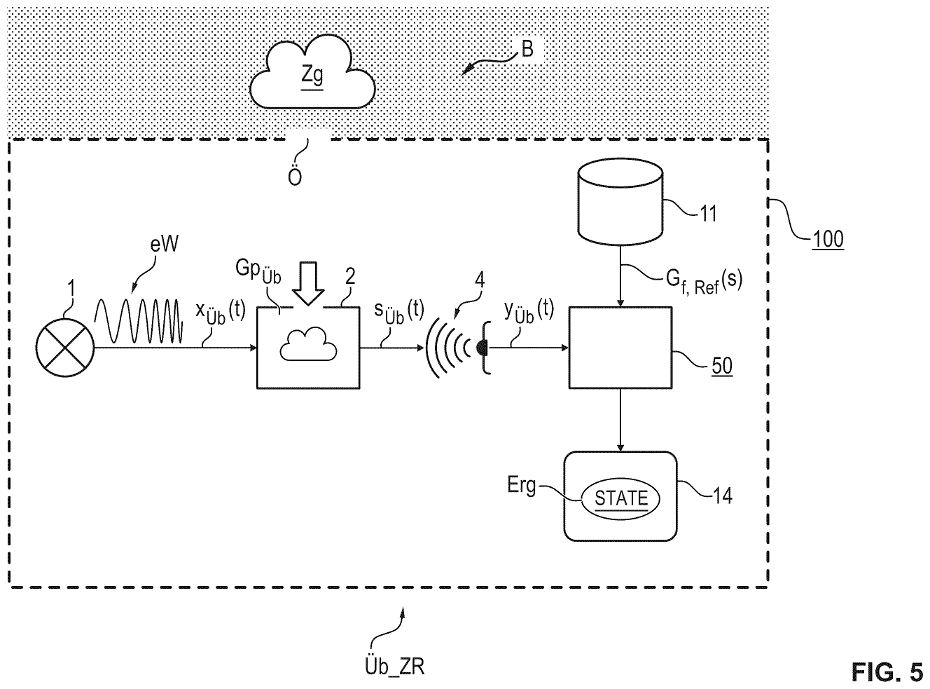

FIG. 3 and FIG. 5 schematically show the gas measuring device 100 according to one embodiment of the configuration example. The gas measuring device 100 comprises a measurement chamber 2 and a radiation source 1.

The measurement chamber 2 is at least temporarily in fluid connection with the spatial region B to be monitored via an opening Ö. This area B can have at least one target gas Zg to be detected. A gas sample Gp_{Ref}, Gp_{Üb} flows through the opening Ö from the area B into the measurement chamber 2, for example by a pump or other fluid delivery unit sucking in the gas sample Gp_{Ref}, Gp_{Üb} and/or by the gas sample Gp_{Ref}, Gp_{Üb} diffusing into the measurement chamber 2.

The radiation source 1 emits radiation eW with a frequency band. In one embodiment, the radiation source 1 emits electromagnetic radiation, in particular infrared radiation, and in another embodiment it emits acoustic radiation (sound or ultrasound). The term “radiation” used below is intended to include in particular electromagnetic radiation in the visible range, infrared range or ultraviolet range as well as sound and ultrasound.

This radiation eW penetrates the measurement chamber 2 at least once, optionally several times. The gas measuring device 100 applies a principle that is well known from the prior art. The target gas or at least one target gas Zg to be detected in the measurement chamber 2 attenuates the radiation eW in a frequency range, which was described with reference to FIG. 1 and FIG. 2. This frequency range generally differs from target gas to target gas and is known in advance. The intensity of the respective attenuation depends on the target gas concentration. The frequency range in which the attenuation takes place usually depends only on the target gas type, but not on the target gas concentration. A detector measures an indicator of the intensity of the incident radiation after the emitted radiation eW has penetrated the measurement chamber 2. This indicator of intensity correlates with the target gas concentration.

In FIG. 3 and FIG. 5, the time-varying intensity of the emitted radiation eW is described by a signal x_{Ref}(t), x_{Üb}(t). This signal x_{Ref}(t), x_{Üb}(t) refers to an area upstream of the measurement chamber 2 and is referred to below as the input signal. The input signal x_{Ref}(t), x_{Üb}(t) is measured or is specified by the control of the radiation source 1. If a control of the radiation source 1 is used as the input signal, the actual intensity may deviate from the control because the radiation source 1 is faulty. In some cases, thanks to the invention, an indication of such a fault in the radiation source 1 can also be detected.

Preferably, the radiation source 1 emits the radiation eW in such a way that the input signal x_{Ref}(t), x_{Üb}(t) is sinusoidal with a frequency that varies over time (frequency sweep signal). In one implementation, the radiation source 1 emits the radiation eW in such a way that the input signal x_{Ref}(t), x_{Üb}(t) is ideally a white noise signal. In reality, a white noise signal can only be approximated. In order to come close to a white noise signal, a broadband noise signal with limited energy is preferably emitted in practice. The term white noise is used in the claims relating to such a broadband noise signal.

The radiation eW completely penetrates the measurement chamber 2 at least once. The intensity of the radiation eW that has penetrated measurement chamber 2 is described by the signal s_{Ref}(t), s_{Üb}(t).

The gas measuring device 100 analyzes the gas sample Gp_{Ref}, Gp_{Üb} in the measurement chamber 2. For the duration of this analysis, the gas sample Gp_{Ref}, Gp_{Üb} is treated as a linear time-invariant system. This assumption is generally justified in particular because neither the gas measuring device 100 itself nor the chemical composition of the gas sample Gp_{Ref}, Gp_{Üb} in the measurement chamber 2 changes significantly while the gas sample Gp_{Ref}, Gp_{Üb} is being analyzed.

The gas measuring device 100 also comprises a detector 4, which is arranged in or on the measurement chamber 2. The detector 4 is excited by incident radiation eW and provides a time-resolved signal. The signal x_{Ref}(t), x_{Üb}(t), which describes the intensity of the radiation eW impinging on the measurement chamber 2, is treated as the input signal for the linear time-variant system just described. The signal that the detector 4 delivers in response to the excitation by the signal s_{Ref}(t), S_{Üb}(t) is treated as the output signal of this system model and is designated as y_{Ref}(t), y_{Üb}(t). These two designations are explained below.

The gas measuring device 100 is used in a reference period Ref_ZR and in a verification period Üb_ZR. It is assumed that the gas measuring device 100 is intact in the reference period Ref_ZR. In the verification period Üb_ZR, the objective is to check whether the gas measuring device 100 is still intact or has a fault—in general: whether its status has changed in a relevant way compared to the status in the reference period Ref_ZR. It is also possible that the reference period Ref_ZR is after the verification period Üb_ZR and therefore it should be determined whether the gas measuring device 100 was intact or faulty in the verification period Üb_ZR.

In particular, the following faults can occur, which are detected by the invention:
A component suddenly fails.
The detector 4 becomes faulty, for example because harmful gases are deposited or due to ageing or a sudden external influence.
The radiation source 1 becomes faulty, for example also due to deposits or ageing or due to a sudden failure.
A wall of the measurement chamber 2 absorbs or scatters radiation eW, which is a particular error if the radiation is to penetrate the measurement chamber 2 several times in order to extend the optical path. One possible cause is that water condenses on a reflective wall.
The power supply for the power source 1 becomes weaker, for example due to ageing of an internal power supply unit or faults in a power supply network.

The output signal obtained by the detector 4 in the reference period Ref_ZR is designated y_{Ref}(t). The output signal from the verification period Üb_ZR is designated y_{Üb}(t). The input signal in the reference period Ref_ZR is designated by x_{Ref}(t), the input signal in the verification period Üb_ZR is designated by x_{Üb}(t). Accordingly, the indicator for the intensity of the radiation eW that impinges on the detector 4 in the reference period Ref_ZR is designated S_{Ref}(t) and the indicator for the intensity of the radiation eW impinging in the verification period Üb_ZR is designated s_{Üb}(t). FIG. 3 to FIG. 9 show whether the representation refers to the reference period Ref_ZR or to the verification period Üb_ZR.

If the gas measuring device 100 is error-free and the input signals x_{Ref}(t) and x_{Üb}(t) match, the two signals s_{Ref}(t) and s_{Üb}(t) and the two output signals y_{Ref}(t) and y_{Üb}(t) ideally match.

The input signal x_{Ref}(t) and the output signal y_{Ref}(t) refer to the same reference time period Ref_ZR and to the same sampling times in this matching reference time period Ref_ZR. Accordingly, the input signal x_{Üb}(t) and the output signal y_{Üb}(t) refer to the same verification period Üb_ZR and to the same sampling times in this matching verification period Üb_ZR.

In the example, the following conditions are created in the reference period Ref_ZR and in the verification period Üb_ZR:

In both time periods Ref_ZR, Üb_ZR, the radiation source 1 emits radiation eW with a frequency band, wherein the intensity of the radiation eW is above a predefined lower threshold value in this frequency band.
The gas sample Gp_{Üb}, which is located in measurement chamber 2 in the verification period Üb_ZR, absorbs the radiation eW in this frequency band up to a specified tolerance in the same way as the gas sample Gp_{Ref} in the reference period Ref_ZR. For example, the two gas samples are chemically very similar.

A signal processing unit 50 receives the input signal x_{Ref}(t), x_{Üb}(t) and the output signal y_{Ref}(t), y_{Üb}(t), processes these signals and delivers a result that includes status information about the status of the gas measuring device 100. This status information refers to the status in the verification period Üb_ZR. In the embodiment according to FIG. 3 and FIG. 5, the signal processing unit 50 is a component of the gas measuring device 100, and the gas measuring device 100 further comprises an output unit 14. The output unit 14 outputs the status information visually and/or acoustically and/or haptically (by vibrations).

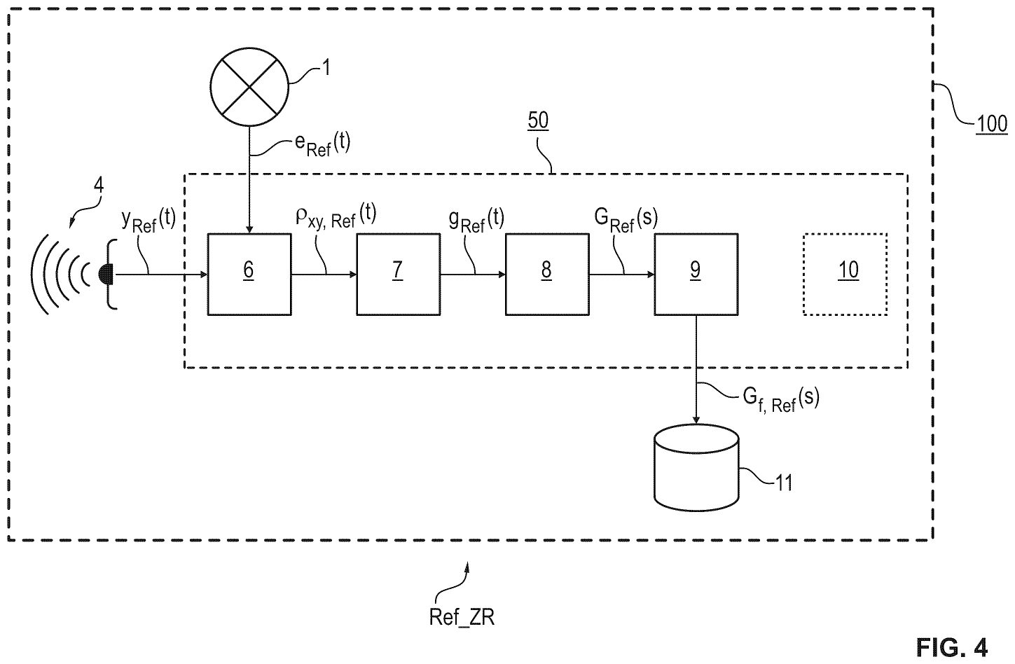

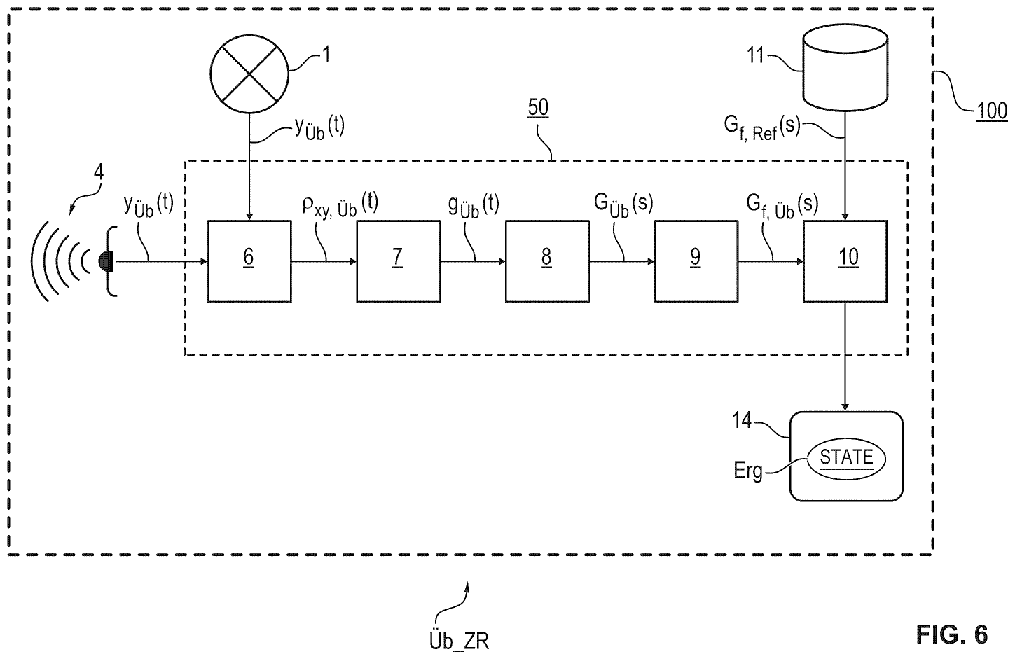

This signal processing unit 50 is described in more detail below, with reference to FIG. 4 and FIG. 6.

In the embodiment example, the signal processing unit 50 comprises at least one processor, a data memory 11 and the following functional components, which are preferably configured as software programs that run on the processor while the gas measuring device 100 is being used:
a cross correlator 6,
an optional smoothing unit 7,
a transformer 8,
an optional filter unit 9 and
an analysis unit 10.

The cross-correlator 6 calculates an indicator for the cross-correlation ρ_{xy,Ref}=ρ_{xy,Ref}(τ) and ρ_{xy,Üb}=ρ_{xy,Üb}(τ) between the input signal x_{Ref}(t), x_{Üb}(t) and the output signal y_{Ref}(t), y_{Üb}(t). The cross-correlation ρ_{xy}(τ) describes the similarity between two signals x(t) and y(t). It is known that the cross-correlation ρ_{xy}(τ) is calculated in the time domain according to the formula

$${ρ}_{xy} ( τ ) = {lim}_{T \to ∞} \frac{1}{T} {{\int }^{+ T / 2}}_{- T / 2} x ( t ) y ( t + τ ) dt . \qquad ( 1 )$$

The output signal y(t) of a linear time-invariant system is known to be related to the input signal x(t) by the so-called impulse response g(t), namely according to the formula

$$y ( t ) = g ( t ) * x ( t ) = {{\int }_{- ∞}}^{+ ∞} g ( u ) x ( t - u ) du . \qquad ( 2 )$$

Here, g(t)*x(t) is the convolution between the two signals g and x.

The cross-correlation ρ_{xy}(τ) is known to be related to the impulse response g(t) according to the formula

$${ρ}_{xy} ( τ ) = {{\int }_{- ∞}}^{+ ∞} g ( u ) {ρ}_{xx} ( τ - u ) du . \qquad ( 3 )$$

Here, ρₓₓ is the autocorrelation of the input signal x(t).

In the following, the index Ref denotes a variable that was generated using the output signal y_{Ref}(t) from the reference time period Ref_ZR, the index Üb denotes a variable that was generated using the output signal y_{Üb}(t) from the review time period Üb_ZR.

Based on the relationships just described, the extractor 7 generates the impulse response g_{Ref}(t) from the cross-correlation ρ_{xy,Ref}(τ) and the impulse response g_{Üb}(t) from the cross-correlation ρ_{xy,Üb}(T).

The transformer 8 transforms the impulse responses g_{Ref}(t) and g_{Üb}(t) from the time domain into the frequency domain, in the embodiment example by means of a Laplace transformation. This Laplace transformation is used to calculate the transfer function G_{Ref}(s) of the impulse response g_{Ref}(t) and the transfer function G_{Üb}(s) of the impulse response g_{Üb}(t).

It is known that the transfer function G(s) in the complex domain of an impulse response g(t) is calculated by a Laplace transformation according to the following calculation rule:

$$G ( s ) = \{ g ( t ) \} = {{\int }_{- ∞}}^{+ ∞} g ( t ) exp ( - s t ) dt \qquad ( 4 )$$

with s=σ+jω, where σ is the real part, j is the imaginary number and ω=2πf is the angular frequency.

In an alternative implementation, the following relationship is used to calculate the transfer functions G_{Ref}(s) and G_{Üb}(s):

$${G}_{Ref} ( s ) = \frac{\{ {Y}_{Ref} ( t ) \}}{\{ {X}_{Ref} ( t ) \}} and {G}_{\ddot{U} b} ( s ) = \frac{\{ {Y}_{\ddot{U} b} ( t ) \}}{\{ {X}_{\ddot{U} b} ( t ) \}} . \qquad ( 5 )$$

In this configuration, the Laplace transforms of the input signals x_{Ref}(t), x_{Üb}(t) and the Laplace transforms of the output signals y_{Ref}(t), y_{Üb}(t) are calculated. This configuration avoids the need to calculate a cross-correlation.

The optional filter unit 9 generates the filtered transfer function G_{f,Ref}(s) from the transfer function G_{Ref}(s) and the filtered transfer function G_{f,Üb}(s) from the transfer function G_{Üb}(s).

The transfer function G_{Ref}(s) or the filtered transfer function G_{f,Ref}(s) from the reference period Ref_ZR is stored in the data memory 11 and read out again from the data memory 11 for checking.

The comparison unit 10 compares the transfer function G_{Üb}(s) from the verification period Üb_ZR with the transfer function G_{Ref}(s) from the reference period Ref_ZR, optionally the two filtered transfer functions G_{f,Üb}(s) and G_{f,Ref}(s) with each other. Ideally, these two transfer functions agree with each other for an intact gas measuring device 100. A deviation above a specified tolerance is an indication of a fault. The comparison unit 10 preferably determines the phase response and the amplitude response. In many cases, the comparison of the phase responses provides an indication of the frequencies and therefore the target gases at which a fault occurs. The comparison of the amplitude responses can be an indication of the target gas concentrations at which a fault occurs.

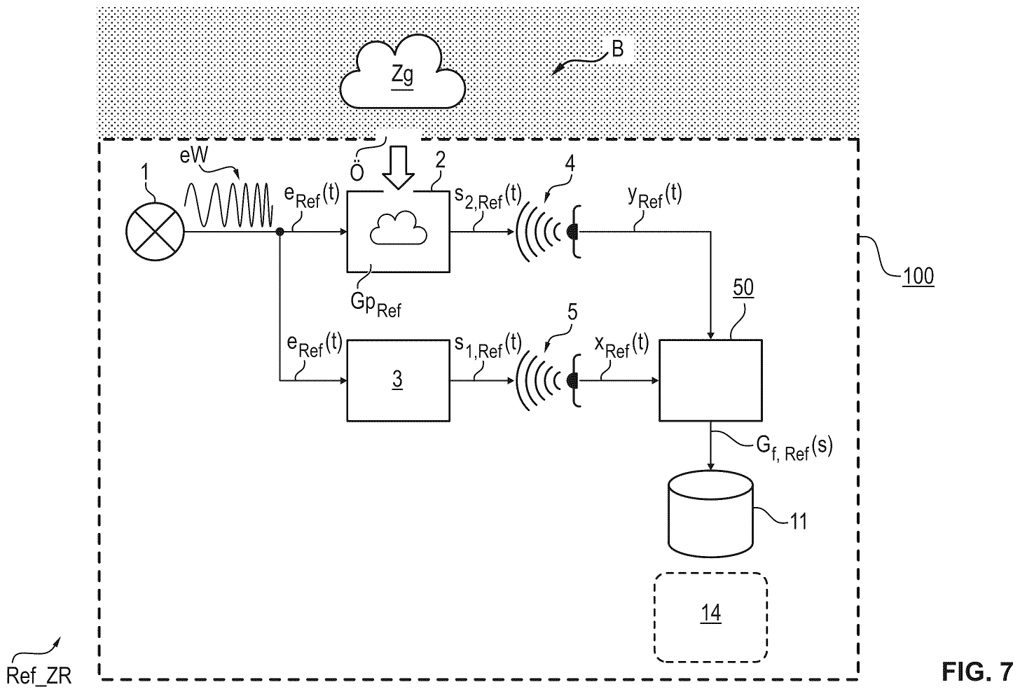

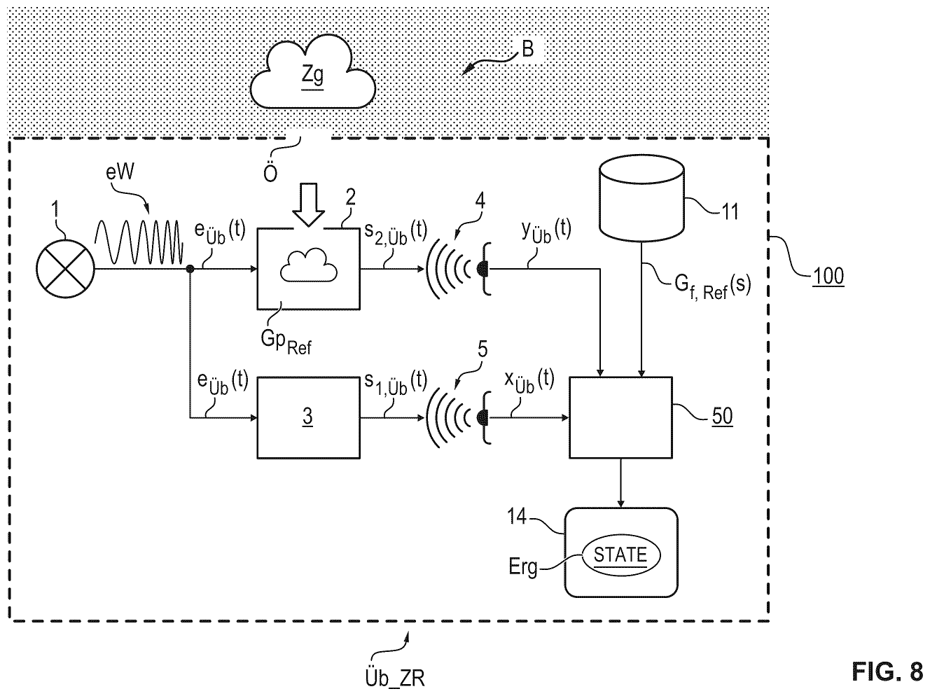

FIG. 7 and FIG. 8 show an alternative embodiment, whereby FIG. 7 again refers to the reference period Ref_ZR and FIG. 8 to the verification period Üb_ZR. Identical reference characters have the same meanings as in FIG. 3 to FIG. 6.

According to this embodiment, the gas measuring device 100 comprises a reference chamber 3 in addition to the measurement chamber 2. The reference chamber 3 is filled with a reference gas Rg, which is free of the target gas or each target gas to be detected. The radiation source 1 emits radiation eW both into the measurement chamber 2 and into the reference chamber 3, and both chambers 2, 3 are arranged parallel to the radiation source 1. The signal that describes the time-varying intensity of the emitted radiation eW before this radiation reaches chambers 2, 3 is designated as e_{Ref}(t) or e_{Üb}(t). The intensity of the radiation eW that has penetrated the measurement chamber 2 and therefore a gas sample Gp_{Ref}, Gp_{Üb} in the measurement chamber 2 at least once is designated by s_{2,Ref}(t) or s_{2,Üb}(t). The intensity of the radiation eW, which has penetrated the reference chamber 3 at least once, is designated by s_{1,Ref}(t) or s_{1,Üb}(t).

Just as in the embodiment shown in FIG. 3 to FIG. 6, the detector 4 also generates an output signal y_{Ref}(t), y_{Üb}(t) in the embodiment shown in FIG. 7 and FIG. 8, which depends on the intensity s_{2,Ref}(t) or s_{2,Üb}(t) of the incident radiation eW. A reference detector 5 generates a signal depending on the incident radiation eW. This signal depends on the intensity s_{1,Ref}(t) or s_{1,Üb}(t) of the radiation eW that has passed through the reference chamber 3 at least once and impinges on the reference detector 5. Alternatively, this signal generated by the reference detector 5 is used as the input signal x_{Ref}(t), x_{Üb}(t). The system model, whose system behavior is calculated, is thus excited with the signal x_{Ref}(t), x_{Üb}(t) of the reference detector 5 and generates the output signal y_{Ref}(t), y_{Üb}(t) in response to this excitation. The further steps in the verification are the same as those described above with reference to FIG. 3 to FIG. 6.

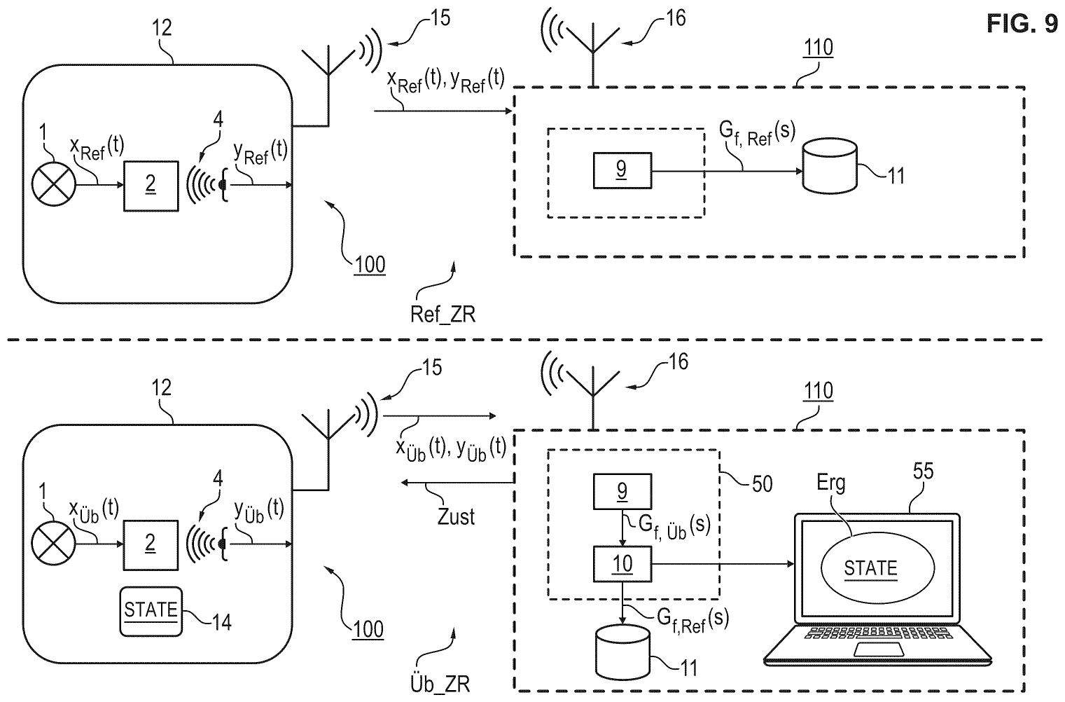

In the embodiment according to FIG. 3 to FIG. 8, the signal processing unit 50 is a component of the gas measuring device 100. FIG. 9 shows an alternative embodiment.

According to this alternative embodiment, the signal processing unit 50 is a component of a computer unit 110, which is spatially remote from the gas measuring device 100 and is at least temporarily in a data connection with it. The data connection can be established by cable or by radio waves. This configuration makes it easier to implement the signal processing unit 50 on a powerful computer. In the implementation form shown, the gas measuring device 100 comprises a communication unit 15 which exchanges messages with a communication unit 16 of the computer unit 110 via radio waves. The gas measuring device 100 transmits the input signal x_{Ref}(t) and the output signal y_{Ref}(t) to the computer unit 110 in the reference time period Ref_ZR and the input signal x_{Üb}(t) and the output signal y_{Üb}(t) to the computer unit 110 in the verification time period Üb_ZR. The analysis unit 10 generates a result Erg with status information STATE. In the example shown, this status information STATE is on the one hand displayed on an output computer 55 of the computer unit 110. On the other hand, the status information STATE is transmitted back to the gas measuring device 100 and output there on the output unit 14.

While specific embodiments of the invention have been shown and described in detail to illustrate the application of the principles of the invention, it will be understood that the invention may be embodied otherwise without departing from such principles.

List of reference characters

| | |
|---|---|
| 1 | Radiation source, emits electromagnetic or acoustic radiation eW into the |
| | measurement chamber 2, the intensity of which is described by the input signal |
| | x_{Ref}(t), x_{Üb}(t) or e_{Ref}(t), e_{Üb}(t) |
| 2 | Measurement chamber, takes up the gas sample Gp_{Ref}, Gp_{Üb} |
| 3 | Reference chamber, takes up the reference gas Rg |
| 4 | Detector at measurement chamber 2, provides the time-resolved output signal y_{Ref}(t) |
| | in the reference period Ref_ZR and the time-resolved output signal y_{Üb}(t) in the |
| | verification period Üb_ZR |
| 5 | Reference detector at reference chamber 3, provides the time-resolved input signal |
| | x_{Ref}(t) in the reference time period Ref_ZR and the time-resolved input signal x_{Üb}(t) |
| | in the verification time period Üb_ZR |
| 6 | Cross-correlator, calculates the cross-correlation ρ_{xy, Ref}(t) between the input signal |
| | x_{Ref}(t) and the output signal y_{Ref}(t) in the reference time period Ref_ZR and the |
| | cross-correlation ρ_{xy, Üb}(t) between the input signal x_{Üb}(t) and the output signal y_{Üb}(t) |
| | in the verification time period Üb_ZR |
| 7 | Extractor, generates the impulse response g_{Ref}(t) from the cross-correlation ρ_{xy, Ref}(t) |
| | in the reference time period Ref_ZR and the impulse response g_{Üb}(t) from the cross- |
| | correlation ρ_{xy, Üb}(t) in the review time period Üb_ZR |
| 8 | Transformer, transforms the impulse response g_{Ref}(t), g_{Üb}(t) from the time domain |
| | into the frequency domain, provides the transformation result G_{Ref}(s), G_{Üb}(s) |
| 9 | Filter unit, generates the filtered transfer function G_{f, Ref}(s) from the transfer function |
| | G_{Ref}(s) and the filtered transfer function G_{f, Üb}(s) from the transfer function G_{Üb}(s) |
| 10 | Comparison unit, compares the two indicators G_{f, Ref}(s), G_{f, Üb}(s) for the system |
| | behavior with each other, provides as a result information on the current status of |
| | the gas measuring device 100 |
| 11 | Data memory in which the indicator G_{f, Ref}(s) for the reference system behavior is |
| | stored in the reference period Ref_ZR |
| 12 | Housing of the gas measuring device 100 |
| 14 | Output unit of the gas measuring device 100 or the computer unit 110, outputs the |
| | status information status of the gas measuring device 100 visually and/or |
| | acoustically and/or haptically |
| 15 | Communication unit of the gas measuring device 100 |
| 16 | Communication unit of the computer unit 110 |
| 50 | Signal processing unit, comprises the cross correlator 6, the smoothing unit 7, the |
| | transformer 8, the optional filter unit 9 and the comparison unit 10, has read access |
| | to the data memory 11, in one embodiment a component of the gas measuring |
| | device 100 and in another embodiment a component of the computer unit 100 |
| 55 | Output computer with screen, displays the status information |
| 100 | Gas measuring device to be tested, comprises the measurement chamber 2, the |
| | detector 4, the radiation source 1, the housing 12, the data memory 11 and, in one |
| | embodiment, the signal processing unit 50 and, in another embodiment, the |
| | communication unit 15 |
| 110 | Remote computer unit, comprising the signal processing unit 50, the output |
| | computer 55 and the communication unit 16, wirelessly connected to the gas |
| | measuring device 100 |
| B | Spatial region to be monitored, can have at least one target gas Zg |
| Erg | Result of the comparison unit 10, comprises the status information |
| e_{Ref}(t) | Intensity of the radiation eW before penetrating the two chambers 2, 3 in the |
| | reference period Ref_ZR |
| e_{Üb}(t) | Intensity of the radiation eW before penetrating the two chambers 2, 3 in the |
| | verification period Üb_ZR |
| eW | Electromagnetic or acoustic radiation, emitted by the radiation source 1 into the |
| | measurement chamber 2, has the intensity x_{Ref}(t), x_{Üb}(t) before passing through the |
| | measurement chamber 2 and the intensity s_{Ref}(t), S_{Üb}(t) after passing through it |
| Gp_{Ref} | Gas sample, flows before or in the reference period Ref_ZR from the area B to be |
| | monitored into the measurement chamber 2 |
| Gp_{Üb} | Gas sample, flows from the area B to be monitored into the measurement chamber 2 |
| | before or during the monitoring period Üb_ZR |
| g_{Ref}(t) | Impulse response in the time domain, is generated in the reference time period |
| | Ref_ZR by the extractor 7 from the cross-correlation ρ_{xy, Ref}(t) |
| g_{Üb}(t) | Impulse response in the time domain, is generated by the extractor 7 from the cross- |
| | correlation ρ_{xy, Üb}(t) in the verification period Üb_ZR |
| G_{Ref}(s) | Transfer function of the system that supplies the output signal y_{Ref}(t) in response to |
| | an excitation by the input signal x_{Ref}(t), describes the system behavior in the |
| | reference time period Ref_ZR, is supplied by the transformer 8 |
| G_{Üb}(s) | Transfer function of the system that supplies the output signal y_{Üb}(t) in response to |
| | an excitation by the input signal x_{Üb}(t), describes the system behavior in the |
| | verification period Üb_ZR, is supplied by the transformer 8 |
| G_{f, Ref}(s) | Filtered transfer function in the frequency domain, supplied by the filter unit 9 in |
| | the reference time period Ref_ZR |
| G_{f, Üb}(s) | Filtered transfer function in the frequency domain, supplied by the filter unit 9 in |
| | the verification period Üb_ZR |
| ρₓₓ(t) | Autocorrelation of the input signal x(t) |
| ρ_{xy, Ref}(t) | Cross-correlation in the time domain between the input signal x_{Ref}(t) and the output |
| | signal y_{Ref}(t) in the reference time period Ref_ZR, calculated by the cross-correlator |
| | 6 |
| ρ_{xy, Üb}(t) | Cross-correlation in the time domain between the input signal x_{Üb}(t) and the output |
| | signal y_{Üb}(t) in the verification period Üb_ZR, calculated by the cross-correlator 6 |
| Ref_ZR | Reference period in which the gas measuring device 100 is intact |
| Rg | Reference gas in reference chamber 3, free of any target gas |
| s_{Ref}(t) | Signal describing the intensity of the radiation eW after penetrating the |
| | measurement chamber 2 in the reference period Ref_ZR |
| s_{Üb}(t) | Signal describing the intensity of the radiation eW after penetrating the |
| | measurement chamber 2 in the verification period Üb_ZR |
| Üb_ZR | Verification period in which the gas measuring device 100 may be faulty |
| x_{Ref}(t) | Time-resolved input signal, describes the intensity of the radiation eW that the |
| | radiation source 1 emits into the measurement chamber 2 in the reference time |
| | period Ref_ZR |
| x_{Üb}(t) | Time-resolved input signal, describes the intensity of the radiation eW that the |
| | radiation source 1 emits into the measurement chamber 2 in the verification period |
| | Üb_ZR |
| y_{Ref}(t) | Time-resolved output signal, supplied by detector 4 in response to the input signal |
| | x_{Ref}(t) in the reference period Ref_ZR |
| y_{Üb}(t) | Time-resolved output signal, supplied by detector 4 in response to the input signal |
| | x_{Üb}(t) in the monitoring period Üb_ZR |
| STATE | Status information -Information about the current status of the gas measuring device |
| | 100 |

What is claimed is:

1. A verification process for verifying a gas measuring device, the gas measuring device comprising: a measurement chamber; a detector; and a radiation source, wherein the measurement chamber is configured to receive a gas sample to be analyzed, the verification process comprising the steps of:
with the radiation source, emitting radiation into the measurement chamber both in a reference period and in a verification period that differs from the reference period, wherein an indicator of an intensity of the emitted radiation before entering the measurement chamber is specified or measured as a respective input signal and at least a part of the emitted radiation at least once penetrates the gas sample in the measurement chamber such that the radiation in the measurement chamber has an intensity that depends on the input signal,
with the detector, generating an output signal depending on an intensity of radiation in the measurement chamber, wherein the measured intensity occurs after the radiation has passed at least once through at least part of the gas sample in the measurement chamber;
calculating in the time domain or in the frequency domain an indicator for a system behavior of a system model, wherein said system model is excited with the input signal and said system model generates the output signal in response to this excitation, wherein the radiation source, the measurement chamber and the detector are intact in the reference period;
comparing a verification system behavior with a reference system behavior, the verification system behavior being a system behavior of said system model that is excited with the input signal in the verification period and generates the output signal in response to this excitation and the reference system behavior being a system behavior of said system model that is excited with the input signal in the reference period and generates the output signal in response to this excitation,
wherein the comparison is performed using the indicator for the system behavior calculated in the verification period and the indicator for the system behavior calculated in the reference period; and
depending on a result of the comparison between the verification system behavior and the reference system behavior, deriving status information about a status of the gas measuring device in the verification period.

2. A verification process according to claim 1, wherein in both the reference period and the verification period, the step of calculating the indicator for the system behavior comprises the step that an indicator of cross-correlation between the input signal and the output signal is calculated.

3. A verification process according to claim 2, wherein in both the reference period and the verification period, the step of calculating the indicator for the system behavior includes the additional steps that a transformation of the cross-correlation into the frequency domain is carried out and that the indicator for the system behavior is determined using the transformation of the cross-correlation into the frequency domain.

4. A verification process according to claim 3, wherein in both the reference period and the verification period the steps are performed that
the radiation source emits the radiation with an input signal in a form of white noise into the measurement chamber,
by using the transformation of the cross-correlation into the frequency domain, the respective transfer function of the system model is calculated when excited by the input signal, and
the respective indicator for the system behavior is calculated using the respective transfer function.

5. A verification process according to claim 1,
wherein the intensity of the emitted radiation in a frequency band is greater than or equal to a lower threshold value and the measurement chamber is filled with a reference gas sample in the reference period and with a verification gas sample in the verification period,
wherein the two gas samples attenuate the radiation in the frequency band to the same extent up to a specified tolerance, and
wherein the respective chemical composition of the two gas samples differ from each other by no more than a predetermined composition tolerance.

6. A verification process according to claim 1,
wherein a target gas set with at least one target gas to be detected is specified,
wherein for every target gas of the target gas set, a frequency band of the radiation emitted by the radiation source covers a respective target gas frequency band,
wherein the target gas absorbs radiation in the respective target gas frequency band more strongly than a predefined lower limit.

7. A verification process according to claim 1,
wherein the verification process is performed using a signal processing unit, the signal processing unit being spatially remote from the gas measuring device; and
wherein the verification process comprises the additional steps of:
in both the reference period and the verification period, transmitting the respective input signal and the respective output signal from the gas measuring device to the signal processing unit;
with the signal processing unit, calculating the indicator for the verification system behavior and the indicator for the reference system behavior;
with the signal processing unit, comparing the verification system behavior with the reference system behavior, the comparison is performed using the verification system behavior indicator and the reference system behavior indicator; and
with the signal processing unit, deriving the status information, and causing the status information to be output in at least one form that can be perceived by a human.

8. A verification process according to claim 1,
wherein the gas measuring device additionally comprises a reference chamber and a reference detector,
wherein the reference chamber is filled with a reference gas which is free of the or each target gas to be detected,
wherein in both the reference period and the verification period, the radiation source emits radiation both into the measurement chamber and into the reference chamber so that emitted radiation penetrates both chambers at least once each,
wherein the measurement chamber and the reference chamber are arranged in parallel with respect to the radiation,
wherein in both in the reference period and in the verification period, the reference detector generates a time-resolved signal, depending on the respective intensity of emitted radiation in the reference chamber, and
wherein as the system model a system model is used which is excited with the signal from the reference detector as the input signal and generates in response to this excitation the output signal from the detector.

9. A verification process according to claim 1,
wherein the detector generates the output signal in both the reference period and the verification period depending on the intensity of the radiation in the measurement chamber and depending on an applied value of a parameter,
wherein several possible values for the parameter are specified,
wherein the process comprises the further performed steps of:
in the verification period, for each possible value of the parameter, calculating the indicator for the verification system behavior, the indicator resulting from the possible parameter value, and comparing the calculated verification system behavior resulting from the possible parameter value with the the reference system behavior,
determining an indicator of a deviation between the verification system behavior resulting from the possible parameter value and the reference system behavior; and
applying a parameter value depending on the indicator of deviation for possible parameter values.

10. A measuring process for measuring a concentration of a target gas, the measuring process comprising the steps of:
providing a gas measuring device, the gas measuring device comprising: a measurement chamber; a detector; and a radiation source;
receiving a gas sample to be analyzed at the measurement chamber;
with the radiation source, emitting radiation into the measurement chamber such that at least a part of the emitted radiation penetrates at least once at least a part of the gas sample in the measurement chamber such that the radiation in the measurement chamber has an intensity that depends on the input signal and on the gas sample;
with the detector, generating an output signal depending on the intensity of the radiation in the measurement chamber, wherein the measured intensity occurs after the radiation has at least once penetrated at least part of the gas sample in the measurement chamber;
measuring a gas concentration as a function of the output signal; and
carrying out a verification process for the gas measuring device, the verification process comprising the steps of:
with the radiation source, emitting radiation into the measurement chamber both in a reference period and in a verification period that differs from the reference period, wherein an indicator of an intensity of the emitted radiation before entering the measurement chamber is specified or measured as a respective input signal and wherein at least a part of the emitted radiation at least once penetrates the gas sample in the measurement chamber such that the radiation in the measurement chamber has an intensity that depends on the input signal,
with the detector, generating an output signal depending on an intensity of radiation in the measurement chamber, wherein the measured intensity occurs after the radiation has passed at least once through at least part of the gas sample in the measurement chamber;
calculating in the time domain or in the frequency domain an indicator for a system behavior of a system model, wherein said system model is excited with the input signal and said system model generates the output signal in response to this excitation,
wherein the radiation source, the measurement chamber and the detector are intact in the reference period;
comparing a verification system behavior, which is a system behavior of said system model that is excited with the input signal in the verification period and generates the output signal in response, with a reference system behavior, which is a system behavior of said system model that is excited with the input signal in the reference period and generates the output signal in response to this excitation,
wherein the comparison is performed using the indicator for the system behavior calculated in the verification period and the indicator for the system behavior calculated in the reference period; and
depending on a result of the comparison between the verification system behavior indicator and the reference system behavior indicator, deriving status information about a status of the gas measuring device in the verification period.

11. A verification device for checking a gas measuring device, wherein the gas measuring device to be checked comprises: a measurement chamber; a detector and a radiation source, wherein the measurement chamber is configured to receive a gas sample to be analyzed, wherein the radiation source is configured to emit radiation into the measurement chamber, wherein the gas measuring device is configured such that at least a part of the emitted radiation penetrates at least once at least a part of the gas sample in the measurement chamber and an intensity of the radiation in the measurement chamber depends on an input signal, wherein the input signal is an indicator of the intensity of the emitted radiation before the radiation enters the measurement chamber, wherein the detector is configured to generate an output signal depending on an intensity of the radiation in the measurement chamber, wherein the measured intensity occurs after the radiation has passed at least once through at least part of the gas sample in the measurement chamber,
the verification device comprising:
a signal processing unit; and
a data connection at least temporarily established between the signal processing unit and the gas measuring device,
wherein the verification device is configured to trigger, both in a reference period and in a verification period that is different from the reference period, the steps of:
with the radiation source, emitting radiation into the measurement chamber;
detecting or measuring the respective input signal as an indicator of the respective intensity of the emitted radiation before the radiation enters the measurement chamber; and
with the detector, generating an output signal, wherein the output signal depends on the intensity of the radiation that occurs after the radiation has penetrated at least part of the gas sample in the measurement chamber,
wherein the radiation source, the measurement chamber and the detector are intact in the reference period,
wherein the signal processing unit is configured to calculate in the time domain or in the frequency domain an indicator of a system behavior of a system model,
wherein said system model is excited with the input signal and this system model generates the output signal in response to this excitation,
wherein the signal processing unit is configured to compare the verification system behavior, the verification system behavior being the system behavior of the system model that is excited with the input signal in the verification period and generates the output signal in response to this excitation, with the reference system behavior, the reference system behavior being the system behavior of the system that is excited with the input signal in the reference period and generates the output signal in response to this excitation,
the signal processing unit being configured to perform the comparison using the indicator for the system behavior calculated in the verification period and the indicator for the system behavior calculated in the reference period; and
wherein the signal processing unit is configured to derive status information about the status of the gas measuring device in the verification period depending on the result of the comparison between the verification system behavior with the reference system behavior.

12. A verification device in accordance with claim 11 in combination with the gas measuring device to provide a measuring arrangement, wherein the verification device is configured to check the gas measuring device.
